(19) United States
(12) Patent Application Publication
(10) Pub. No.: US 2021/0169681 A1
(43) Pub. Date: Jun. 10, 2021

(54) CONTROLLED TEMPERATURE WATER BOTTLE

(71) Applicant: Robert Dempsey, Kent, WA (US)

(72) Inventor: Robert Dempsey, Kent, WA (US)

(21) Appl. No.: 16/704,371

(22) Filed: Dec. 5, 2019

(51) Int. Cl. A61F 7/00

(52) U.S. Cl. CPC A61F 7/007; A61F 7/0085; A61F 2007/0094; A61F 2007/0054; A61F 2007/0086

(57) ABSTRACT

An illustrated view of a water bottle for providing therapy to a patient is presented. The water bottle is made of rubber, though the hot water bottle may be of other suitable material to hold water without leaking and to allow the temperature of the water held internally to be felt externally. The hot water bottle is described as holding and heating hot water, but one could also utilize the device and the contemplations of the device to use cool water and chilling the water, adding tepid water and keeping the water at an in between temperature, etc.

### FIELD OF THE INVENTION

This invention relates to apparatus for holding liquid. More particularly, it relates to apparatus to control the temperature of the liquid being held.

### BACKGROUND

Devices for applying heat or cold, such as a heat pack or cold pack, to areas of the human body are well known. Known ice packs, for example, may comprise sealed enclosures which contain particulate matter, water, glycol, a mixture thereof or other fluid medium which are pre-chilled in a refrigerating device and the chilled or, in some cases, frozen pack then applied to an area of the skin of a human or other mammalian body to treat and/or control inflammation, bruising and/or infection and to promote healing and/or to relieve pain.

However, such cold pack devices have disadvantageous aspects and are of limited use since they tend to be too intense in their cooling effect when first applied and have been found to induce frostbite. Moreover, the ice packs lose much of their effectiveness because they warm up rather quickly through heat exchange with the body, thereby providing a constantly changing temperature at the areas of contact.

Other devices in which the heat exchange with the body, and hence the therapeutic treatment, is prolonged are known. These other known devices with prolonged effectiveness include flexible sealed enclosures through which a chilled or heated fluid medium is caused to flow by pumping the fluid medium through the device. Such devices are known in the art by various descriptions such as garments, pipings, bandages, pads, wraps, cuffs and the like, but in each case the device generally comprises a flexible sealed enclosure (hereinafter a ‘thermal pack’) which can be applied and secured to an area of the body to be treated. A chilled or heated fluid medium can be caused to flow through the enclosure to provide a cooling or heated therapeutic treatment.

In relation specifically to cold therapy treatment, or cryotherapy as it is known, equipment to circulate fluid through a thermal pack generally comprises a chiller unit supplying cold fluid to a thermal pack by way of circulation through insulated hoses. More simplistic (and cheaper) systems operate by raising and lowering a water-ice tank to fill and then empty a thermal pack via a single hose.

These systems should not be confused with the cold packs referred to above such as gel filled flexible enclosures which are placed in a freezer and then applied to the treatment area of the body without any circulation or ingress or egress of fluid relative to the pack.

An effective therapy requires the maintenance of a constant even temperature at a desirable level over the treatment area. The various cooling techniques presently in use fail satisfactorily to maintain a constant temperature on the desired body areas or do so only with the use of expensive and unnecessarily complex equipment.

In addition to the requirement of maintaining a constant temperature on the selected areas, there is also a need for a thermal pack that will comfortably and securely fit the contours of the area being treated as well as a need for providing portability of the cooling (or heating) unit.

Application of the thermal pack to an area of the body, e.g. the knee, causes it to fold and crease creating restrictions which reduce or cut off the flow of fluid and causes “dead areas” with little or no fluid movement which results in loss of temperature control at that point. Accordingly, there is a need for ensuring uninterrupted flow and dispersion of fluid over the complete surface of the thermal pack.

### BRIEF DESCRIPTION OF THE DRAWINGS

FIG. 1A is an illustrated view of an exemplary hot water bottle.

FIG. 1B is an illustrated view of a control panel of the exemplary hot water bottle shown in FIG. 1A.

### DETAILED DESCRIPTION

Reference will now be made in detail to the present preferred embodiments, examples of which are illustrated in the accompanying drawings. All terms in the plural shall also be taken as singular and vice-versa. Further, any reference to he shall also be applicable to she and vice-versa.

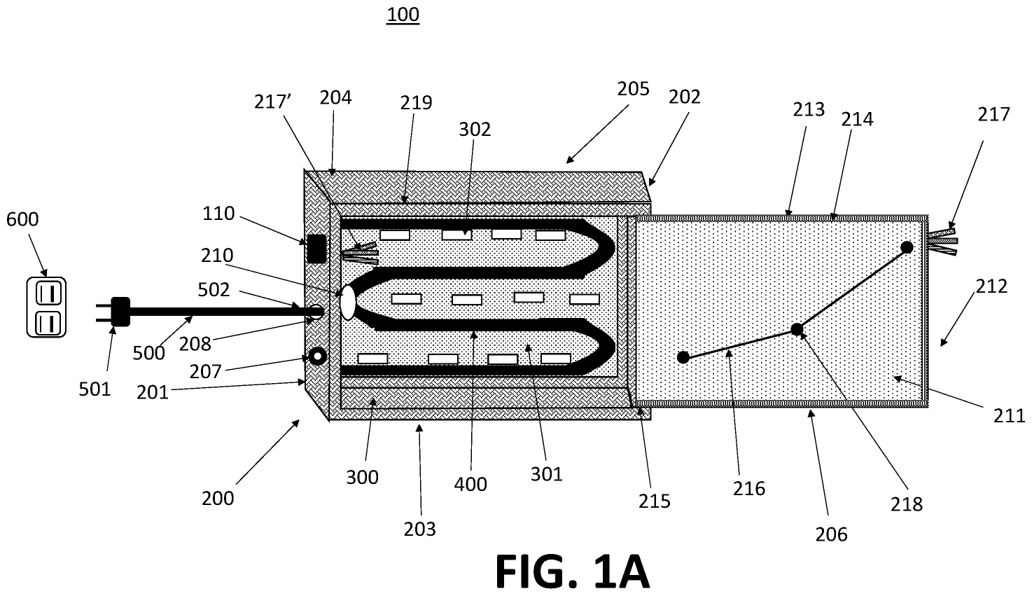

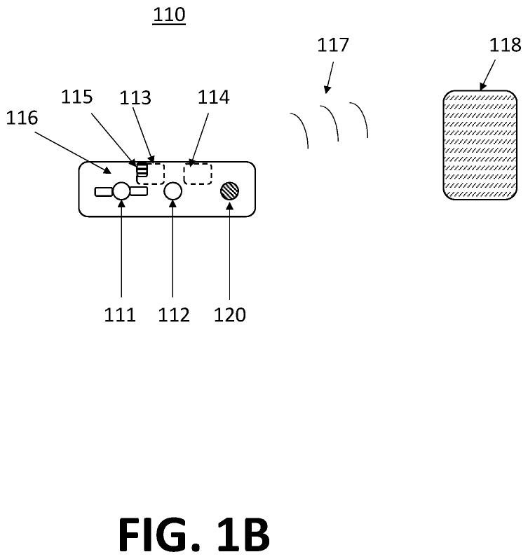

Referring to FIG. 1A and FIG. 1B, an illustrated view of a water bottle 100 for providing therapy to a patient is presented. The water bottle is made of rubber, though the hot water bottle may be of other suitable material to hold water without leaking and to allow the temperature of the water held internally to be felt externally. The hot water bottle is described as holding and heating hot water, but one could also utilize the device and the contemplations of the device to use cool water and chilling the water, adding tepid water and keeping the water at an in between temperature, etc.

The hot water bottle 100 has an outside 200, interior 300 and an energy source 600. The outside 200 has a front 201, a back 202, a first side 203, a second side 204, a bottom 205 and a top 206.

The interior 300 has a bottom 301. A heat strip 400 is securely coupled to the bottom 301 of the interior 300. The heat strip 400 is preferably a single strip that winds through the interior 301 of the interior 300. The bottom 301 of the interior 300 is preferably made of an insulating material.

The interior 300 further has a plurality of pad spacers 302 securely coupled to the bottom 301 of the interior 300. The pad spacers 302 are configured to be equally spaced to further secure the heating strip 400 in a desired location on the bottom 301 of the interior 300 of the hot water bottle 100. The pad spacers 302 are preferably made of a rubber material.

The pad spacers 302 spaced apart approximately two inches apart to help the heat strip 400 to stay in a desired position. The heat strip 400 are glued to the bottom to fix the location of the heat strip 400. The rubber in the pad spacers 302 is used to help prevent the heat strip 400 inside from touching the top 205.

The preferred embodiment of the exemplary hot water bottle 100 has sixteen (16) rubber pad spacers 302 on the interior 300 of the hot water bottle 100. Preferably the rubber pad spacers 302 are spaced two inches apart. The number and spacing of the rubber pad spacers may be any number and spaced any distance.

The bottom 301 of the interior 300 of the hot water bottle 100 further has a felt material for protecting of bottom 301 from contacting the heat strip 400. This helps keep the temperature at a predetermined temperature by helping reduce heat loss. The rubber pad spacers 302 further helps to assure heated water is being circulated.

The front 201 of the outside 200 of the hot water bottle 100 has a water intake port 207, an electric cord port 208 and a control panel 110. The water intake port 207 is configured to be coupled to a water source, such as a hose, allowing water to be entered into the interior 300 of the hot water bottle 100.

The control panel 110 has a temperature control 111, a temp display 112, a computing device 113 and a wireless transceiver 114. The computing device 113 and wireless transceiver 114 are coupled to an inside 116 of the control panel 110. The computing device 113 further has a memory unit 115.

The control panel 110 is configured to adjust and set a desired temperature of the water stored in the interior 300 of the hot water bottle 100 as well as display a current temperature of the water stored in the interior 300 and a set temperature determined at the temperature control 111. The control panel 110 is preferably a touchscreen.

Optionally and/or additionally, when the computing device 113 of the control panel 110 determines a temperature of the water has exceeded a predetermined temperature, the computing device 113 determines an alarm sound stored in the memory unit 114 of the computing device 113 and sends the alarm sound to play to a speaker 120 when an abnormal condition arises. The abnormal condition is preferably a power failure but may be any other determined conditions such as the temperature being below a second threshold.

When the temperature is determined to be greater than the desired temperature, the computing device 113 sends a signal is to reduce the heat being produced by the heat strip 400. When the temperature of the water contained in the hot water bottle 100 is determined by the computing device 113 to be less than the desired temperature set at the temperature control 111, then the computing device 113 sends a signal to increase the temperature of the heat strip 400 thus increasing the temperature of the water contained in the hot water bottle 100.

The thermostat control 111 is preferably a manual thermostat coupled to the control panel 110 but may be wirelessly connected through the wireless transceiver 114 via a wireless signaling protocol 118 to a wireless computing device 118, such as a smart phone, a computer, etc. The wireless signaling protocol may be consistent with any wireless technology, including, but not limited to, Bluetooth (IEEE 802.15), WiFi (IEEE 802.11), etc.

The electric cord port 208 of the front 201 of the outside 200 is configured to receive a power cord 500.

The power cord 500 has a first end 501 and a second end 502. The first end 501 of the power cord 500 is removably coupled to a energy source 600. The energy source 600 is preferably an AC/DC source. The second end 502 of the power cord 500 is threaded through the electric cord port 208 and is coupled to a thermostat 210. The thermostat 210 is configured to be coupled to the heat strip 400 configured on the interior 300 of the hot water bottle 100. The thermostat 210 is further coupled to the computing device 113 of the control panel 110 to regulate the temperature of the heat strip 400 and thus the water contained on the interior 300 will have a temperature determined by the setting of the temperature control 111.

The power cord 500 electrically couples the hot water bottle 100 to the energy source 600. The energy source 600 and control panel 110 are for coupling an instrument to provide power and to control the temperature of the water contained in the interior 300 of the hot water bottle 100.

The top 206 of the hot water bottle 100 has an interior side 211 and an exterior side 212. The interior side 211 has an edge 213 where the edge 213 has a coupling device 214, such as Velcro®, adhering compound, etc., that provides a water proof seal when the top 206 is in a closed position, thus preventing water contained in the interior 300 of the hot water bottle 100 from exiting the interior 300 of the hot water bottle 100.

The outside 200 has an edge 219 where the edge 219 is the parameter of the interior 300 of the hot water bottle 100. A first end 215 of the top 206 is hingedly coupled to the edge 219 substantially near the back 202 of the outside 200 of the hot water bottle. When the top 206 is position in a closed condition the edge 213 of the interior side 211 of the top 206 is removably and sealingly coupled to the edge 219 of the outside 200 of the hot water bottle 100 by the coupling device 214.

The interior side 211 of the top 206 further has a temperature indicator 216. The temperature indicator 216 has a plurality of heat sensors 218. The heat sensors 218 are preferably three (3) in number equally spaced along the temperature indicator 216.

Each of a plurality of wires 217 are coupled to each of the plurality of heat sensors 218, such that each of the heat sensors 218 are individually coupled to transmit information to the computing device 113. A second end of the plurality of wires 217′ are coupled to the computing device 113 of the control panel 110 where the computing device 113 determines the temperature of the water contained in the interior 300 of the hot water bottle 100. The computing device 113 further determines whether to change the temperature of the heat strip 400 based on the determined temperature of the water contained on the interior 300 of the hot water bottle 100.

The exterior side 212, when the top 206 is in a closed condition, is removably coupled to a patient.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A controlled temperature device, the device comprising:
an interior, the interior having a bottom;
a heat strip, the heat strip coupled to the bottom of the interior;
a plurality of padded spacers, the padded spacers being coupled to the bottom of the interior;
an outside, the outside having a front and a back;
a water intake port, the water intake port being coupled to the front of the outside;
an electric cord port, the electric cord port being coupled to the front of the outside;
a control panel, the control panel having an inside, a temp display and a temperature control;
a computing device, the computing device having a memory unit, and wherein the computing device being coupled to the inside of the control panel;
a power cord, the power cord having a first end and a second end, wherein the second end being threaded through the electric cord port, and wherein the first end being coupled to an energy source, wherein the energy source being coupled to a thermostat coupled to the heat strip and wherein the energy source being coupled to the computing device;
a top, the top having an interior side and exterior side;
a temperature indicator, the temperature indicator having a plurality of heat sensors; and
a plurality of wires, each of the plurality of wires being coupled to one of the plurality of sensors, and wherein each of the plurality of wires being coupled to the computing device.

2. The controlled temperature device of claim 1, wherein the control panel further comprising a wireless transceiver, wherein the wireless transceiver being coupled to the computing device.

3. The controlled temperature device of claim 2, wherein the wireless transceiver being consistent with an IEEE 802.11 (WiFi) protocol.

4. The controlled temperature device of claim 1, wherein the control panel is a touchscreen.

5. The controlled temperature device of claim 6, wherein the thermostat control is in communication with an app on a computing device.

6. The controlled temperature device of claim 1, wherein the controlled temperature device is rectangular.

7. The controlled temperature device of claim 1, further comprising an alarm for alerting when an abnormal condition arises.

8. The controlled temperature device of claim 7, wherein the abnormal condition is a power failure.

9. The controlled temperature device of claim 7, further the control panel further comprising a speaker wherein the alarm is auditable via the speaker.

10. The controlled temperature device of claim 7, wherein the abnormal condition is a temperature above a threshold.

11. The controlled temperature device of claim 7, wherein the abnormal condition is a temperature below a threshold.

12. The controlled temperature device of claim 1, wherein the bottom being felt.

13. The controlled temperature device of claim 1, wherein the plurality of heat sensors being three (3) in number.

14. The controlled temperature device of claim 1, wherein the plurality of wires being three (3) in number.
